(19) United States
(12) Patent Application Publication
(10) Pub. No.: US 2013/0235348 A1
(43) Pub. Date: Sep. 12, 2013

(54) OPHTHALMOLOGIC APPARATUS

(71) Applicant: CANON KABUSHIKI KAISHA, Tokyo (JP)

(72) Inventor: Shohhei Kamada, Tokyo (JP)

(73) Assignee: CANON KABUSHIKI KAISHA, Tokyo (JP)

(21) Appl. No.: 13/782,298

(22) Filed: Mar. 1, 2013

(30) Foreign Application Priority Data
Mar. 8, 2012 (JP) 2012-051451

(51) Int. Cl. A61B 3/00

(52) U.S. Cl. CPC A61B 3/0008
USPC 351/221; 351/246

(57) ABSTRACT

In order to detect a deterioration status of an imaging light source in an accurate manner, an ophthalmologic apparatus includes a light intensity detection unit configured to detect a light intensity of light emitted from the imaging light source, a determination unit configured to determine a status of the imaging light source on the basis of the light intensity detected by the light intensity detection unit and a light emitting time of the imaging light source, and a display control unit configured to display, on a display unit, the status of the imaging light source on the basis of a determination result by the determination unit.

### BACKGROUND OF THE INVENTION

1. Field of the Invention

The present invention relates to an ophthalmologic apparatus that is configured to control an imaging light intensity at the time of imaging an eye to be inspected.

2. Description of the Related Art

In a conventional ophthalmologic apparatus such as a fundus camera, a light source such as a xenon tube is used as a light source for imaging a fundus of an eye to be inspected. In the case of using such a light source as an imaging light source, in general, a part of imaging light is received by a light receiving element such as a photodiode and an output from the light receiving element is integrated by an integration circuit including an operational amplifier and a capacitor when imaging an image. An output from the integration circuit is then compared with a reference voltage, and when the output from the integration circuit is equal to or larger than the reference voltage, the emission is stopped, to thereby control the light intensity to be a constant value.

It has been known that the light source, such as the one described above, is generally deteriorated with time. The deterioration becomes apparent in a form of non-emission at the time of imaging an image or a light intensity deterioration in which the light intensity is reduced even when the same voltage and current are applied as before the deterioration. The non-emission leads to a failure of the imaging, and a frequency of the non-emission is increased with time. The light intensity deterioration, once deteriorated, is continued to be deteriorated with time thereafter, which disables an imaging that requires a large light intensity (such as a autofluorescence imaging).

In order to cope with the above-mentioned problems, an apparatus has been known, which includes a measurement unit for measuring an emission energy from an emission start to an emission end for each imaging, and detects a deterioration status of the light source from data of an integrated energy that indicates how much the light source has been used (see, for example, Japanese Patent Application Laid-Open No. H06-047000).

In the apparatus such as the one disclosed in Japanese Patent Application Laid-Open No. H06-047000, an accurate model is required, which indicates the deterioration status of the light source with respect to the integrated emission energy. However, it has been known that the emission light source such as the xenon tube generally has a large fluctuation in the quality in a manufacturing process. In addition, the deterioration status of the light source is represented by a complicated system that is determined by various factors such as temporal variation of the light source when not being used and storage and usage environment, as well as the emission energy. Therefore, it is considered to be considerably difficult to create an accurate deterioration model that can predict the deterioration status from the emission energy only.

When there is a discrepancy between a deterioration status predicted from the model and an actual status, not only the function itself becomes meaningless, failing to perform the detection, but also an adverse effect may be caused, such as a case in which it is determined that the light source is deteriorated in spite that it is not deteriorated, so that a user replaces the light source with a new one.

### SUMMARY OF THE INVENTION

In order to solve the above-mentioned problems, an ophthalmologic apparatus according to an exemplary embodiment of the present invention includes: an imaging light source; a light intensity detection unit configured to detect a light intensity of light emitted from the imaging light source; a determination unit configured to determine a status of the imaging light source on the basis of the light intensity detected by the light intensity detection unit and a light emitting time of the imaging light source; and a display control unit configured to display, on a display unit, the status of the imaging light source on the basis of a determination result by the determination unit.

Further, a method of controlling an ophthalmologic apparatus according to another exemplary embodiment of the present invention includes: detecting a light intensity of light emitted from an imaging light source; determining a status of the imaging light source on the basis of the light intensity detected in the detecting and a light emitting time of the imaging light source; and displaying, on a display unit, the status of the imaging light source on the basis of a determination result in the determining.

The ophthalmologic apparatus according to the present invention accumulates a relationship between the imaging light intensity and the light emitting time as data indicating the deterioration status of the light source, and evaluates the deterioration status of the light source from a temporal variation of the data. The deterioration status is actually measured for each imaging, and hence the deterioration status of the light source can be detected without being affected by an individual difference of the light source itself, a deterioration with time when not being used, and a deterioration due to storage and usage environment, which have been error factors in a prediction of the deterioration status performed in the apparatus disclosed in Japanese Patent Application Laid-Open No. H06-047000.

Further features of the present invention will become apparent from the following description of exemplary embodiments with reference to the attached drawings.

### BRIEF DESCRIPTION OF THE DRAWINGS

FIG. 1 is a configuration diagram of a fundus camera according to a first embodiment of the present invention.

FIG. 2 is an electric circuit diagram of a xenon tube driving circuit and a light intensity detection unit.

FIG. 3A is a graph showing a relationship between a light emitting time, an output of an integration circuit, and a deterioration evaluated value according to the first embodiment.

FIG. 3B is a graph showing a temporal variation of the deterioration evaluated value with respect to an integrated operation time according to the first embodiment.

FIG. 4 is a graph showing waveforms of output voltages of the integration circuit in a normal mode and a non-emission mode.

FIG. 5 is a flowchart illustrating a procedure of determining a deterioration status of a light source according to the first embodiment.

FIG. 6 is a flowchart illustrating a procedure of acquiring an image.

FIG. 7A is a graph showing a relationship between a light emitting time, an output of an integration circuit, and a deterioration evaluated value according to a second embodiment of the present invention.

FIG. 7B is a graph showing a temporal variation of the deterioration evaluated value with respect to an integrated operation time according to the second embodiment.

FIG. 8 is a flowchart illustrating a procedure of determining a deterioration status of a light source according to the second embodiment.

### DESCRIPTION OF THE EMBODIMENTS

### First Embodiment

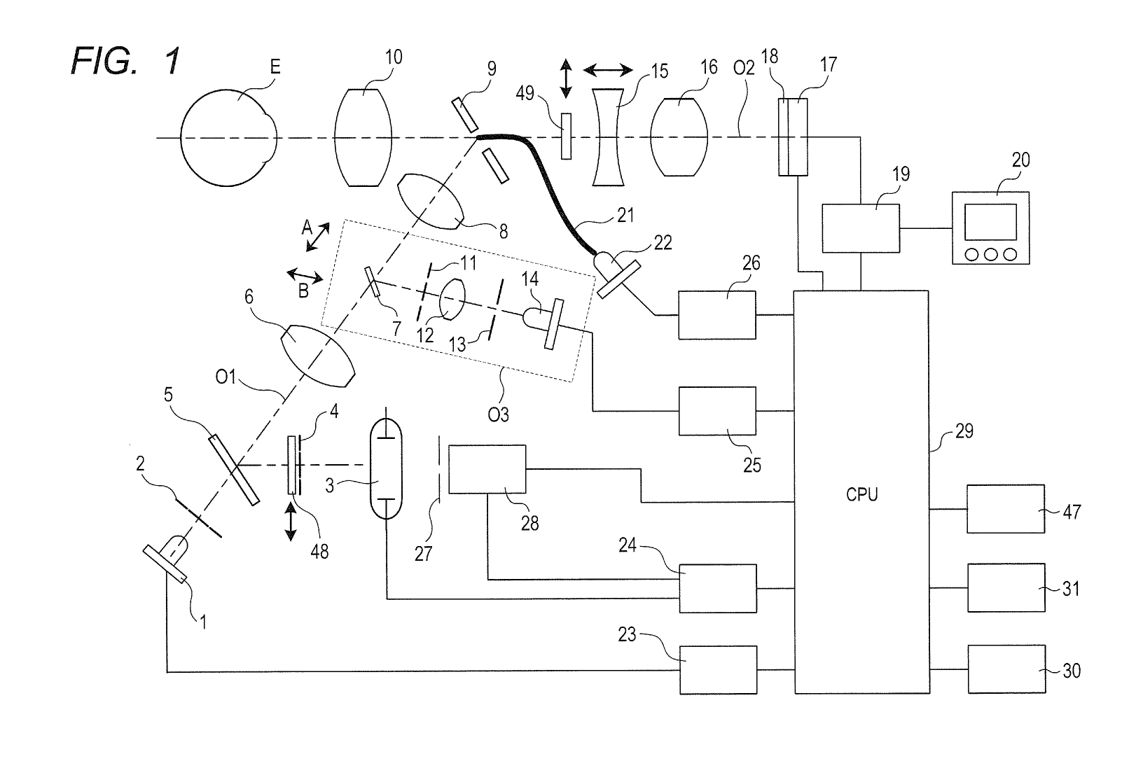

Exemplary embodiments of the present invention are described in detail below with reference to FIGS. 1 to 5.

FIG. 1 is a configuration diagram of a fundus camera according to a first embodiment of the present invention. The fundus camera includes an infrared light emitting diode (LED) 1 that is a light source for monitoring infrared light, a xenon tube 3 that is a light source configured to emit visible light for imaging, an aperture 2 having a ring-shaped opening and arranged ahead of the infrared LED 1, an aperture 4 having a ring-shaped opening and arranged ahead of the xenon tube 3, and a dichroic mirror 5 that transmits the infrared light and reflects the visible light. These components are arranged in this order of description on an optical path extending from the xenon tube 3 to an objective lens 10. A relay lens 6, a mirror 7, a relay lens 8, and a mirror 9 with a hole are sequentially arranged ahead of the dichroic mirror 5, thus constituting a fundus illumination optical system O1. An excitation filter 48 for a autofluorescence imaging is arranged between the aperture 4 and the dichroic mirror 5, which is retractable to an outside of an optical axis by a driving system (not shown), so that the excitation filter 48 is retracted to the outside of the optical axis when performing a color imaging. The xenon tube 3 corresponds to an imaging light source according to the present invention.

An aperture 11, a lens 12, a focusing index 13, and an infrared LED 14 that is a focusing index light source are arranged in a reflection direction of the mirror 7, thus constituting a focusing-index projecting optical system O3. The focusing-index projecting optical system O3 is configured to move in a direction A in FIG. 1 in conjunction with a focusing lens 15. When imaging a still image, the focusing-index projecting optical system O3 moves in a direction B in FIG. 1 by a driving system (not shown) to be retracted from the fundus illumination optical system O1.

The focusing lens 15, an imaging lens 16, and an imaging unit 17 are arranged on an optical axis in a transmission direction of the mirror 9 with a hole, thus constituting a fundus imaging optical system O2. An output of the imaging unit 17 is sequentially coupled to an image signal processing unit 19 and a display unit 20. An infrared LED 22 that is a positioning index light source is coupled to the mirror 9 with a hole through an optical fiber 21. A notch filter 49 for a autofluorescence imaging is arranged between the mirror 9 with a hole and the focusing lens 15, which is retractable to the outside of the optical axis by a driving system (not shown), so that the notch filter 49 is retracted to the outside of the optical axis when performing a color imaging.

A light intensity detection unit 28 is arranged behind the xenon tube 3, which is capable of receiving a part of an emission light beam from the xenon tube 3 through an aperture 27.

The infrared LED 1 is connected to an LED driving circuit 23, the xenon tube 3 for imaging is connected to a xenon tube driving circuit 24, the infrared LED 14 is connected to an LED driving circuit 25, and the infrared LED 22 is connected to an LED driving circuit 26. The LED driving circuit 23, the xenon tube driving circuit 24, the LED driving circuit 25, the LED driving circuit 26, the light intensity detection unit 28, the imaging unit 17, the image signal processing unit 19, an operation unit 30, a recording unit 31, and a memory 47 are connected to a central processing unit (CPU) 29.

Further, a filter 18 is arranged on the imaging unit 17, in which three colors of red (R), green (G), and blue (B) are arranged in a mosaic shape on respective pixels of the imaging unit 17. An R filter can transmit light from the red light to the infrared light. Each pixel has a sensitivity to any one of R light, G light, and B light, and an R pixel further has a sensitivity to the infrared light.

When monitoring the infrared light, the image signal processing unit 19 generates monochrome movie data by using an output of the R pixel, and outputs the movie to the display unit 20. On the other hand, when performing a color imaging, the image signal processing unit 19 generates a color still image by using outputs of the R, G, and B pixels, and when performing the autofluorescence imaging, the image signal processing unit 19 generates a monochrome still image from image processing by using the outputs of the R, G, and B pixels, and records the generated still image in the recording unit 31 via the CPU 29.

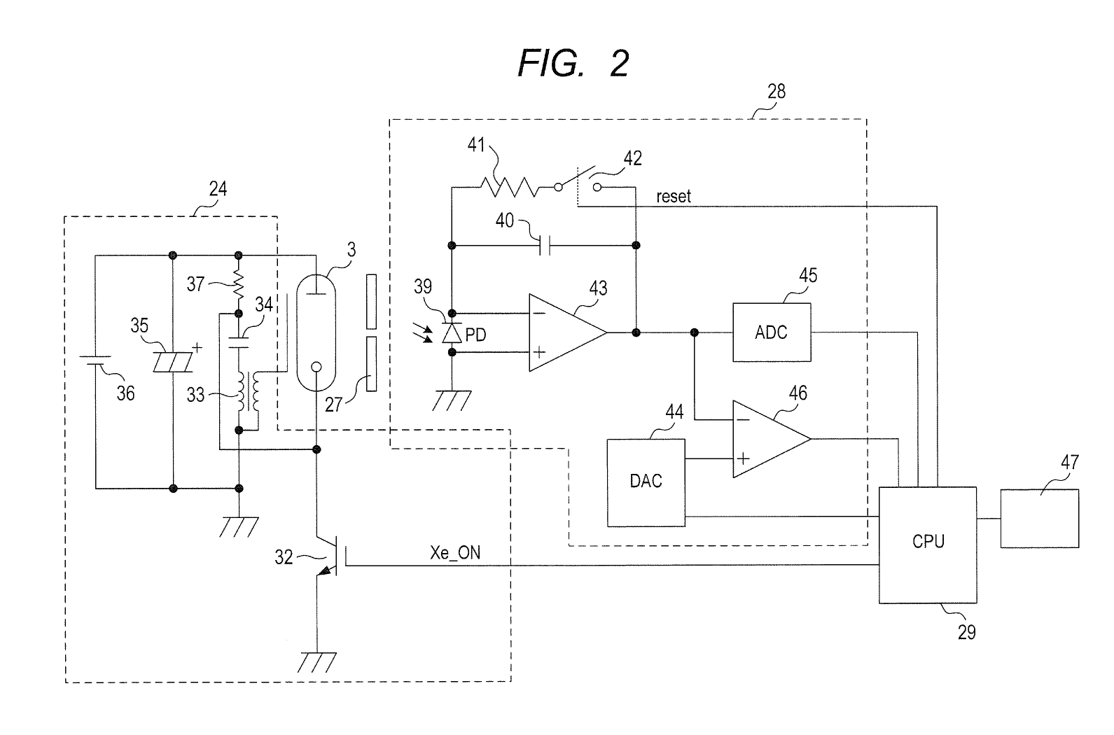

FIG. 2 is a configuration diagram of electric circuits of the xenon tube driving circuit 24 and the light intensity detection unit 28. The xenon tube driving circuit 24 includes an insulated gate bipolar transistor (IGBT) 32, a main capacitor 35, a power source 36, a resistor 37, a trigger capacitor 34, and a trigger transformer 33, and the main capacitor 35 is charged to a high voltage (for example, 300 volts) by the power source 36. The trigger capacitor 34 is also charged through the resistor 37. With this circuit configuration, when the CPU 29 sets a Xe ON signal to High, the IGBT 32 is switched ON, and the trigger capacitor 34 is discharged first so that a current flows to a first winding of the trigger transformer 33. This generates a high voltage on a second winding of the trigger transformer 33, and the xenon tube 3 is triggered. When a current flows from the main capacitor 35 to the xenon tube 3, an emission of the xenon tube 3 is started. After the emission is started, when the CPU 29 sets the Xe ON signal to Low, the IGBT 32 is switched OFF and the current to the xenon tube 3 is blocked, so that the emission is stopped.

The light intensity detection unit 28 includes an integration circuit including a photodiode 39, an integration capacitor 40, a reset resistor 41, an analog switch 42, and an operational amplifier 43. The light intensity detection unit 28 constitutes a light intensity detection unit in the present invention, which is configured to measure an imaging light intensity of imaging light emitted from an imaging light source. When the CPU switches on the analog switch 42, charges of the integration capacitor 40 can be reset through the reset resistor 41.

A digital-to-analog (D/A) converter 44 outputs a reference voltage for stopping the emission of the xenon tube 3 and a reference voltage for resetting the integration capacitor 40. A configuration of the D/A converter 44 for outputting the reference voltage and the like corresponds to an imaging light intensity setting unit in the present invention, which is configured to set an imaging light intensity target value as a target value of imaging light with which an eye to be inspected is irradiated when acquiring an image. An output of the D/A converter 44 is coupled to an input of a comparator 46 together with an output of the operational amplifier 43, so that an output voltage of the integration circuit and an output voltage of the D/A converter 44 can be compared with each other.

An output of the comparator 46 is coupled to the CPU 29. When the output voltage of the integration circuit is lower than the output voltage of the D/A converter 44, a High signal is output from the comparator 46, and in a reverse case, a Low signal is output. The comparator 46 functions as an imaging light intensity control unit in the present invention, which is configured to control, by controlling a light emitting time of the imaging light source in the above-mentioned manner, the imaging light intensity so that the actual imaging light intensity becomes substantially equal to the imaging light intensity target value.

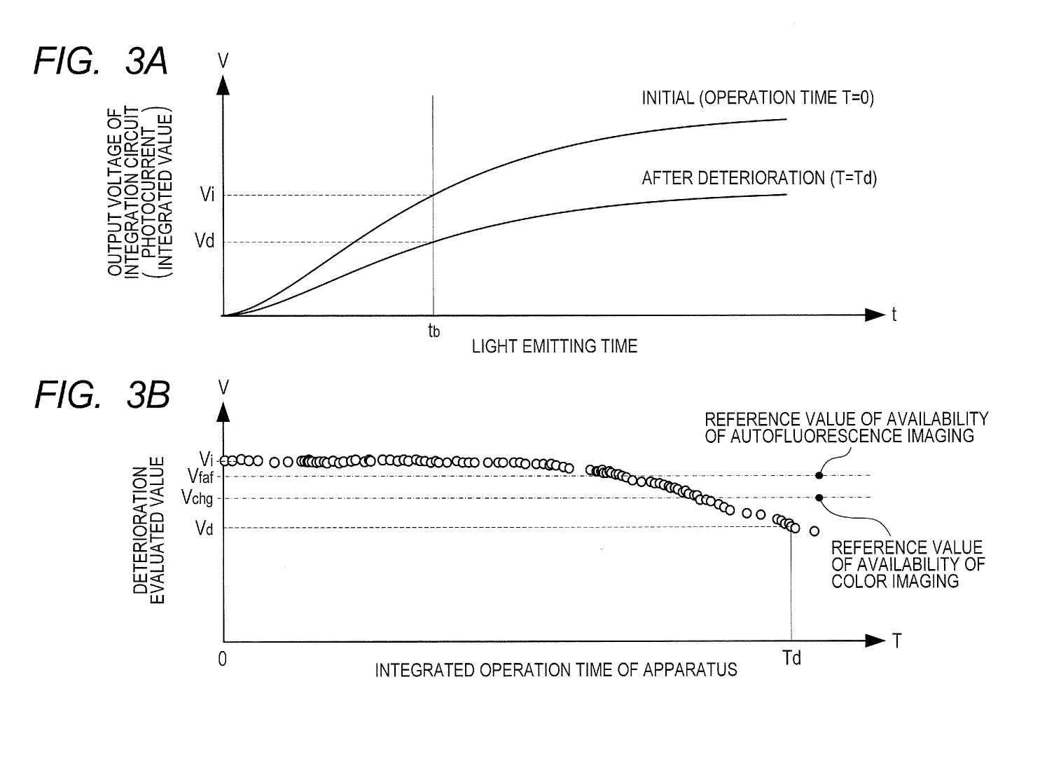

FIG. 3A is a graph obtained by plotting the output voltage of the integration circuit (photocurrent integrated value) with respect to the light emitting time for imaging before the light source is deteriorated and after the light source is deteriorated. As an example that represents the deteriorated light source, a status of the light source after a time T_{d} has elapsed as an operation time of the apparatus is shown in the graph. The time T_{d} in this example is a time in units of year, which is long enough for a user to recognize the deterioration of the light source. The CPU 29 acquires the output voltage of the integration circuit via an analog-to-digital (A/D) converter 45. The output voltage of the integration circuit after the lapse of a reference time t_{b} since the start of the emission is set to an evaluation value measured by the light intensity detection unit or a deterioration evaluated value. With respect to a deterioration evaluated value Vᵢ in an initial condition in which the light source is not deteriorated, the deterioration evaluated value measured when the light intensity is decreased due to a usage over years takes a value as low as V_{d}. The CPU 29 calculates the deterioration evaluated value from the output voltage of the integration circuit acquired for each imaging, and stores the calculated deterioration evaluated value in the memory 47. Because the light intensity of the imaging light source is controlled by the light emitting time, the emission may be stopped before reaching the reference time t_{b} depending on the setting of the light intensity when acquiring an image, and the deterioration evaluated value may not be collected. However, data corresponding to such cases are ignored. In the first embodiment, the reference time t_{b} is set to t_{b}=2 ms which is short enough compared to the normal light emitting time and at which the emission is stabilized.

FIG. 3B is a graph obtained by plotting a temporal variation of the deterioration evaluated value with respect to an integrated operation time of the apparatus. A value of the deterioration evaluated value stored in the memory 47 is decreased from the initial value Vᵢ with the deterioration of the light source due to the usage over years. A reference deterioration evaluated value V_{faf} is set in advance in the apparatus. The reference deterioration evaluated value V_{faf} is a value obtained by adding a margin of +5% to the deterioration evaluated value in a deterioration status in which the light intensity is managed to reach a light intensity for the autofluorescence imaging, which is the maximum emission light intensity of the apparatus, with a full emission. When the deterioration evaluated value is below this value, and the light source is further deteriorated by the margin, it means that the emission performance required for the autofluorescence imaging is lost from the light source. Further, a deterioration evaluation reference value V_{chg} is set in the apparatus. The deterioration evaluation reference value V_{chg} is obtained by adding a margin of +5% to the deterioration evaluated value in a critical deterioration status in which the maximum imaging light intensity for a color imaging mode can be obtained.

Because the emission performance required for the color imaging is low enough compared to the autofluorescence imaging, a relation of V_{faf}>V_{chg} is established.

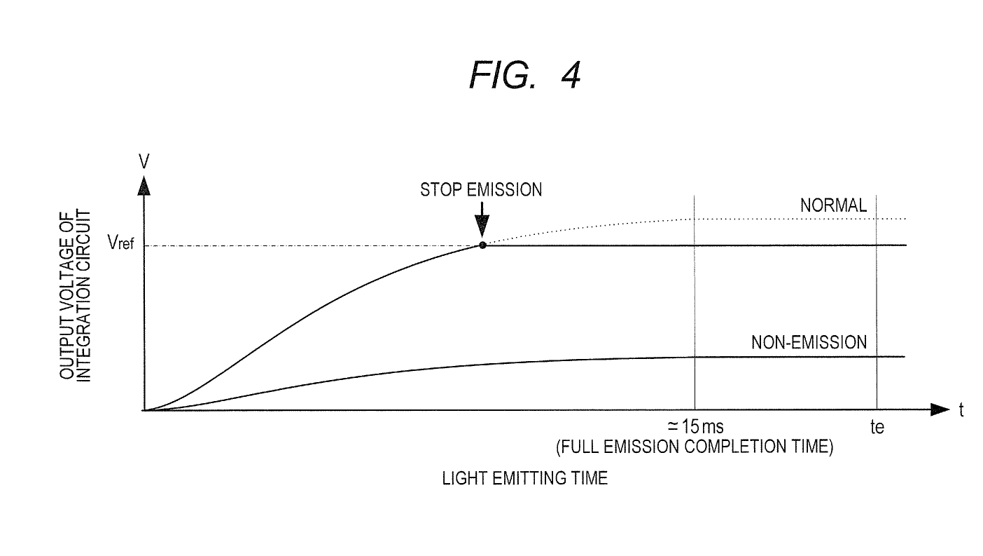

FIG. 4 is a graph showing the output voltages of the integration circuit with respect to the light emitting time in a normal mode and a non-emission mode. The reference deterioration evaluated value V_{ref} is the output of the D/A converter 44, and when the output voltage of the integration circuit exceeds the reference deterioration evaluated value V_{ref}, the IGBT 32 is switched OFF and the emission is stopped. The light emitting time is about 15 ms when the emission stop is not performed by the control on the xenon tube 3 (full emission), and if the emission stop is not performed, the output voltage becomes constant in about 15 ms. A non-emission detection time tₑ, which is longer than the full light emitting time, is set in advance in the apparatus. When the comparator 46 does not output a Low signal even after the lapse of the non-emission detection time tₑ since the start of the emission, the CPU 29 determines the imaging as a non-emission, and displays a failure of the imaging due to the non-emission on the display unit 20. The CPU 29 further records the non-emission in the memory 47.

The above-mentioned memory 47 in which a relationship between the light source light emitting time and the light intensity detection result is recorded as a time-light intensity record and a module area in the CPU 29 that detects a degree of deterioration (which is described later) of the imaging light source from the change of the time-light intensity record constitute a detection unit in the present invention. The degree of deterioration over years is determined on the basis of the above-mentioned evaluation value. Further, the determination of the non-emission described above is performed by a module area that functions as a non-emission detection unit.

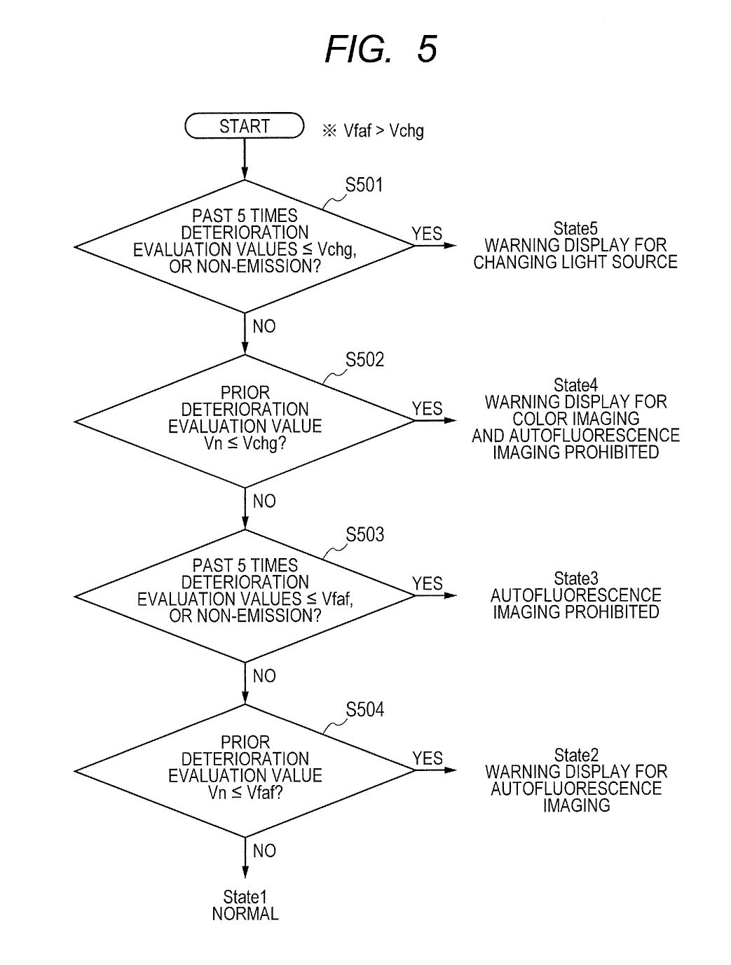

FIG. 5 is a flowchart for determining the deterioration status of the light source on the basis of the deterioration evaluated value and the non-emission detection result according to the first embodiment. The CPU 29 calculates the deterioration evaluated value for each imaging, reads the previous deterioration evaluated value and information on the non-emission from the memory 47, and determines the deterioration status following the determination flowchart illustrated in FIG. 5. The deterioration status includes five levels from State 1 to State 5, and the apparatus performs a prohibition of a specific imaging mode and a warning display on the display unit 20 in accordance with each deterioration status of the light source. Such a deterioration status is defined as the degree of deterioration of the imaging light source in the present invention. According to the present invention, the status of the light source is determined on the basis of the light intensity from the imaging light source and the light emitting time of the imaging light source detected in the above-mentioned manner, and the determination result is notified to the user in various forms to be described later. This determination is performed by a module area in the CPU 29 that performs as a determination unit. This determination unit also includes a module area that functions as a recording control unit that records the light emitting time in the memory 47 for each emission of the imaging light source. In addition, in the present invention, the status of the imaging light source is obtained from the determination result determined by the determination unit. The obtained status is displayed on the display unit 20 in an effective display form selected from forms such as an integrated value of the light emitting time of the xenon tube, information on whether or not the xenon tube should be replaced, and the number of emissions obtainable until the replacement. These display forms also include a message saying “the required light intensity cannot be obtained for the autofluorescence imaging”, as well as the message saying the replacement of the xenon tube, and this selection and instruction to the display unit is performed by a module area that functions as a display control unit in the CPU 29.

Further, when the forms of the display unit and the like are defined more clearly, the display unit 20 for performing the warning display corresponds to a deterioration degree notifying unit, and a module area for prohibiting the imaging corresponds to an imaging prohibiting unit.

When all deterioration determination steps are cleared, the deterioration status is determined to be State in which there is no deterioration, and the warning display, the imaging prohibition, and the like are not performed.

When it falls into a condition of Step S504 in which the latest deterioration evaluated value is below V_{faf}, the deterioration status is determined to be State 2 in which the autofluorescence imaging cannot be performed if the light intensity is deteriorated further, in other words, even when the autofluorescence imaging mode is selected, the selected mode cannot be performed. When the light source is used continuously without being replaced, the CPU issues a warning that the autofluorescence imaging cannot be performed, by displaying it on the display unit 20.

When it falls into a condition of Step S503 in which the past five deterioration evaluated values are the non-emission or below the reference deterioration evaluated value V_{faf}, the deterioration status is determined to be State 3 in which the autofluorescence imaging cannot be performed in a stable manner. The CPU 29 displays a message saying that the autofluorescence imaging mode cannot be performed due to the deterioration of the light source on the display unit 20, and performs a control by the imaging prohibiting unit to disable a selection of the autofluorescence imaging when selecting a mode. Further, in this case, the CPU 29 causes the display control unit to display a display form indicating the prohibition of the autofluorescence imaging on the display unit 20.

When it falls into a condition of Step S502 in which the latest deterioration evaluated value is below V_{chg}, the deterioration status is determined to be State 4 in which not only the autofluorescence imaging but also the color imaging cannot be performed if the light intensity is deteriorated further. The CPU 29 displays a message saying that the autofluorescence imaging cannot be performed, and if the light source is used continuously without a replacement, there may be a problem in the color imaging on the display unit 20, and further disables the selection of the autofluorescence imaging.

When it falls into a condition of Step S501 in which the past five deterioration evaluated values are the non-emission or below the reference deterioration evaluated value V_{chg}, the deterioration status is determined to be State 5 in which the light source is considerably deteriorated so that the imaging cannot be performed in a stable manner. The CPU 29 displays a message saying that the quality of the acquired image cannot be guaranteed due to the deterioration of the light source and a message prompting the replacement of the light source on the display unit 20.

Although not described in a separate embodiment, for example, the timing of displaying the deterioration of the light source may be determined by a prediction on the basis of the accumulation of the deterioration evaluated value stored in the memory 47 and a deterioration prediction expression, rather than determined on the basis of a time when the deterioration evaluated value becomes below the reference value V_{faf} or V_{chg}. Further, although the apparatus according to the first embodiment is configured to perform the color imaging and the autofluorescence imaging, if the apparatus is an ophthalmologic imaging apparatus having a specific imaging mode as well as the color imaging mode and the autofluorescence imaging mode, a deterioration evaluated value unique to the specific imaging mode can be set in the same manner as the above-mentioned reference deterioration evaluated value V_{faf}, so that influence of the deterioration is displayed or the prohibition of the imaging is performed for each imaging mode. In addition, in terms of various parameters for the light intensity, for example, when the apparatus is an ophthalmologic imaging apparatus that is configured to adjust the light intensity in response to an ISO sensitivity, it may be determined whether or not the imaging can be performed with respect to a deterioration status of each ISO sensitivity, and the warning display and the prohibition of the imaging may be performed accordingly.

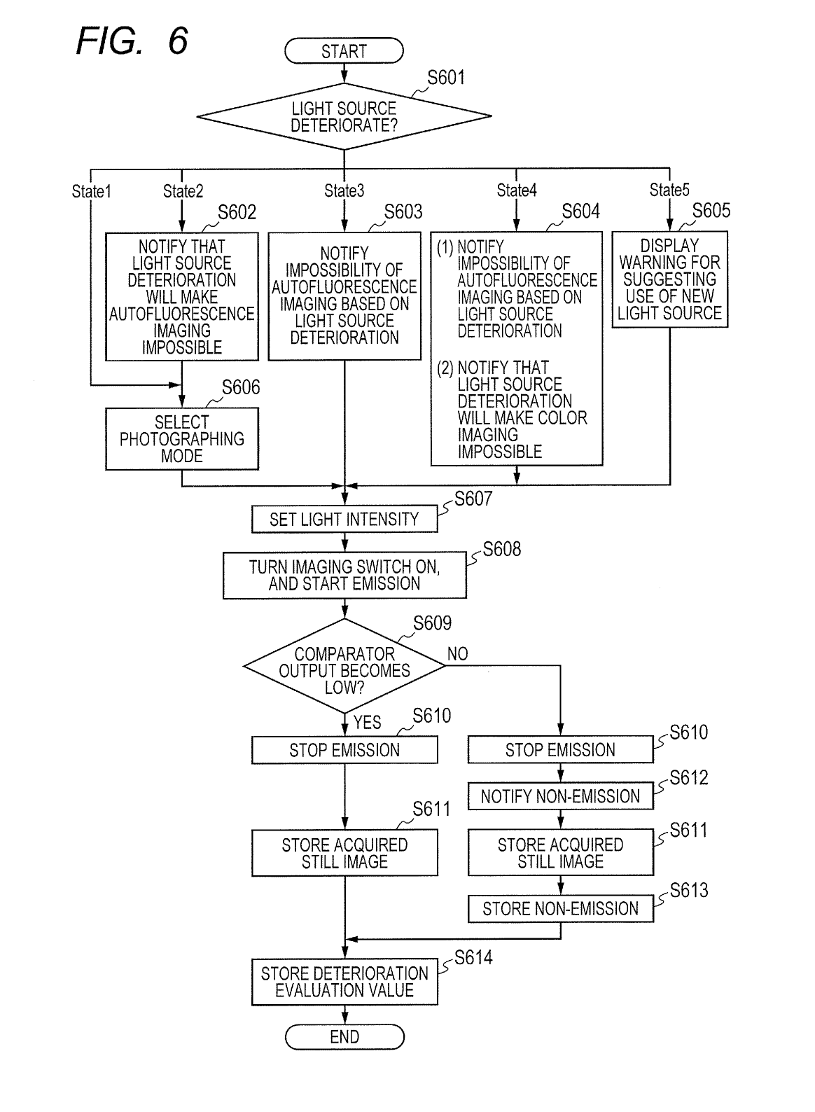

FIG. 6 is a flowchart illustrating a flow of the imaging. The flow of the imaging is described with reference to FIG. 6.

Before starting an imaging, in Step S601, the CPU reads the deterioration evaluated value and the non-emission record from the memory 47, and performs the determination of the light source deterioration status.

When the deterioration status is determined to be State 1 indicating no deterioration, the process control proceeds to Step S606.

When the deterioration status is determined to be State 2, a message saying that a continuous usage will disable the autofluorescence imaging due to the deterioration of the light source is displayed on the display unit 20, and the process control proceeds to Step S606 to select the imaging mode.

When the deterioration status is determined to be State 3, a message saying that the autofluorescence imaging cannot be performed due to the deterioration of the light source is displayed on the display unit 20, and the process control proceeds to Step S607 by skipping Step S606.

When the deterioration status is determined to be State 4, messages saying that the autofluorescence imaging cannot be performed due to the deterioration of the light source and that a further continuous usage may affect the color photography are displayed on the display unit 20, and the process control proceeds to Step 607 by skipping Step 606.

When the deterioration status is determined to be State 5, because the quality of the acruired image cannot be guaranteed due to the advance of the deterioration of the light source, a warning that prompts the replacement of the imaging light source is displayed on the display unit 20, and the process control proceeds to Step S607 by skipping Step S606.

A transition to Step S606 is made only in the cases of State 1 and State 2 in which the autofluorescence imaging can be still performed. In Step S606, the user selects the color imaging or the autofluorescence imaging by operating a mode switch (not shown) of the operation unit 30. When the color imaging is selected, the excitation filter 48 for the autofluorescence imaging is retracted from the optical axis.

In Step S607, a light intensity adjustment value is set by an operation of a light intensity adjustment switch (not shown) of the operation unit 30. The CPU 29 calculates emission light intensity on the basis of the set imaging mode and a light intensity adjustment value, and sets a reference voltage V_{ref} to the comparator 46 via the D/A converter 44. The light intensity adjustment switch also constitutes a part of the imaging light intensity setting unit.

Subsequently, alignment of the fundus camera and an eye E to be inspected is performed by using a fundus image of the eye E to be inspected illuminated by the infrared LED 1 that is the light source for monitoring the infrared light and displayed on the display unit 20, and a positioning index image projected on a cornea of the eye E to be inspected by the infrared LED 22 that is the positioning index light source. Further, focusing is performed by an index image of the infrared LED 14 that is the focusing index light source. When the alignment and the focusing are completed, the user starts the imaging by pressing an imaging switch (not shown) of the operation unit 30 (Step S608). In order to make a transition from the infrared monitoring mode to the still image acquiring mode, the CPU 29 turns off the infrared LED 1, the infrared LED 22, and the infrared LED 14. The CPU 29 retracts the fundus imaging optical system O2 from the optical axis of the fundus illumination optical system O1, and when the autofluorescence imaging is selected, inserts the notch filter 49 for the autofluorescence imaging into the optical axis.

In Step S608, the CPU 29 releases the reset of the integration circuit by switching the analog switch 42 OFF, and the CPU 29 sets the Xe ON signal to High to switch the IGBT 32 ON, to thereby trigger the xenon tube 3 and start the emission.

In Step S609, the CPU 29 stands by until the output of the comparator 46 becomes Low. When the output of the comparator 46 is not changed to Low even after an elapse of the non-emission detection time tₑ since the start of the emission, Step S612 is inserted after the emission stop in Step S610. In Step S612, the CPU 29 displays a message saying that the imaging has failed due to an occurrence of the non-emission on the display unit 20. Further, Step 613 is inserted after storing an acquired still image in Step S611. In Step S613, the CPU 29 records the imaging non-emission in the memory 47.

Returning to Step S609, when the output of the comparator 46 is Low or when the non-emission detection time tₑ has elapsed since the start of the emission, the process control proceeds to Step S610, where the CPU 29 sets the Xe ON signal to Low to switch the IGBT 32 OFF, so that the emission of the xenon tube 3 is stopped.

Thereafter, in Step S611, the image signal processing unit 19 generates a still image in accordance with the imaging mode from the output of the imaging unit 17, stores the still image in the recording unit 31, and then the process control proceeds to Step S614. In Step S614, the CPU 29 calculates the deterioration evaluated value on the basis of the output from the A/D converter 45, and stores the deterioration evaluated value in the memory 47.

### Second Embodiment

A second embodiment of the present invention is different from the first embodiment only in the calculation of the deterioration evaluated value and the determination of the deterioration status, and therefore, descriptions on the other parts are omitted.

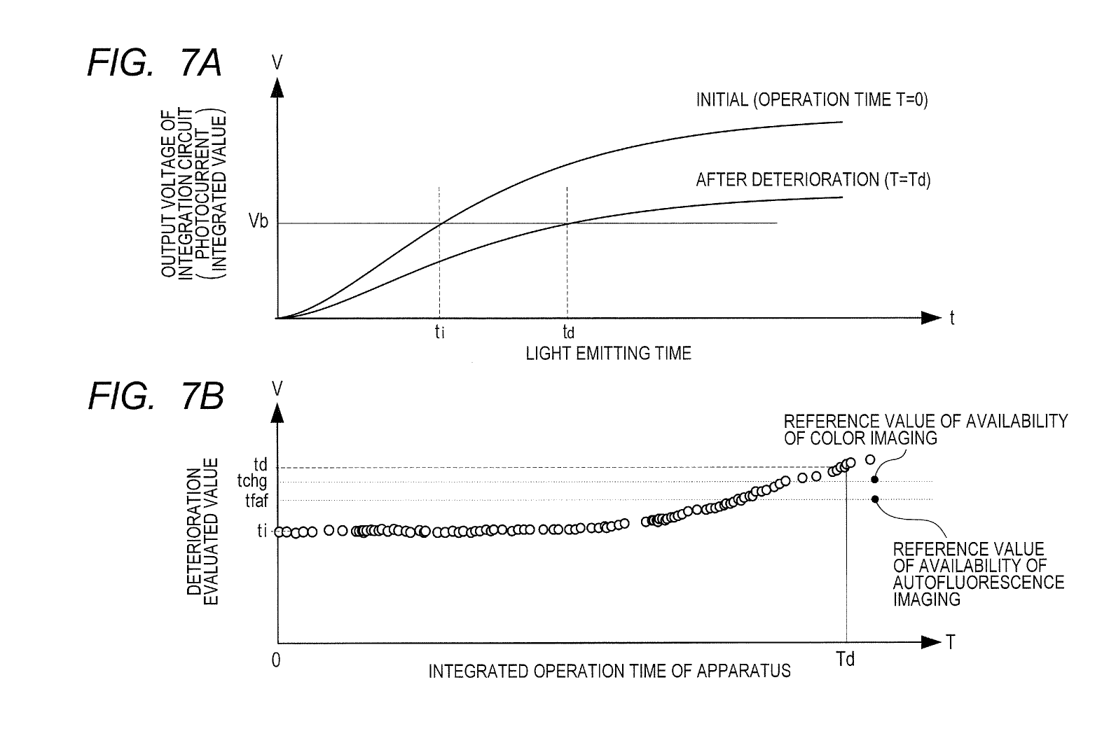

FIG. 7A is a graph obtained by plotting the output voltage of the integration circuit (photocurrent integrated value) with respect to the light emitting time for imaging before the light source is deteriorated and after the light source is deteriorated. As an example that represents the deteriorated light source, a status of the light source after a time T_{d} has elapsed as an operation time of the apparatus is shown in the graph. The CPU 29 acquires the output voltage of the integration circuit. In this example, a time from the start of the emission until the output of the integration circuit reaches the evaluation reference voltage V_{b} is set as the deterioration evaluated value. With respect to a deterioration evaluated value tᵢ in the initial condition in which the light source is not deteriorated, the deterioration evaluated value measured when the light intensity is increased due to a usage over years is a value as long as t_{d}. The CPU 29 calculates the deterioration evaluated value from the output voltage of the integration circuit acquired for each imaging, and stores the calculated deterioration evaluated value in the memory 47. The emission of the imaging light source is configured to be stopped when the light intensity reaches a target imaging light intensity set before the imaging, and hence the emission may be stopped before reaching the reference voltage V_{b} depending on the target imaging light intensity, and the deterioration evaluated value may not be collected. However, data corresponding to such cases are ignored. By setting the reference voltage V_{b} to a low value, such a situation is avoided to the least level.

FIG. 7B is a graph obtained by plotting a temporal variation of the deterioration evaluated value with respect to an integrated operation time of the apparatus. A value of the deterioration evaluated value stored in the memory 47 is increased from an initial value tᵢ with the deterioration of the light source due to the usage over years. A reference deterioration evaluated value t_{faf} is set in advance in the apparatus. The reference deterioration evaluated value t_{faf} is a value obtained by adding a margin of +5% to the deterioration evaluated value in a deterioration status in which the light intensity is managed to reach a light intensity for the autofluorescence imaging, which is the maximum emission light intensity of the apparatus, with a full emission. When the deterioration evaluated value exceeds this value, and the light source is further deteriorated by the margin, it means that the emission performance required for the autofluorescence imaging is lost from the light source. Further, a deterioration evaluation reference value t_{chg} is set in the apparatus. The deterioration evaluation reference value t_{chg} is obtained by adding a margin of +5% to the deterioration evaluated value in a critical deterioration status in which the maximum imaging light intensity for a color imaging mode can be obtained.

Because the emission performance required for the color imaging is low enough compared to the autofluorescence imaging, a relation of t_{chg}>t_{faf} is established.

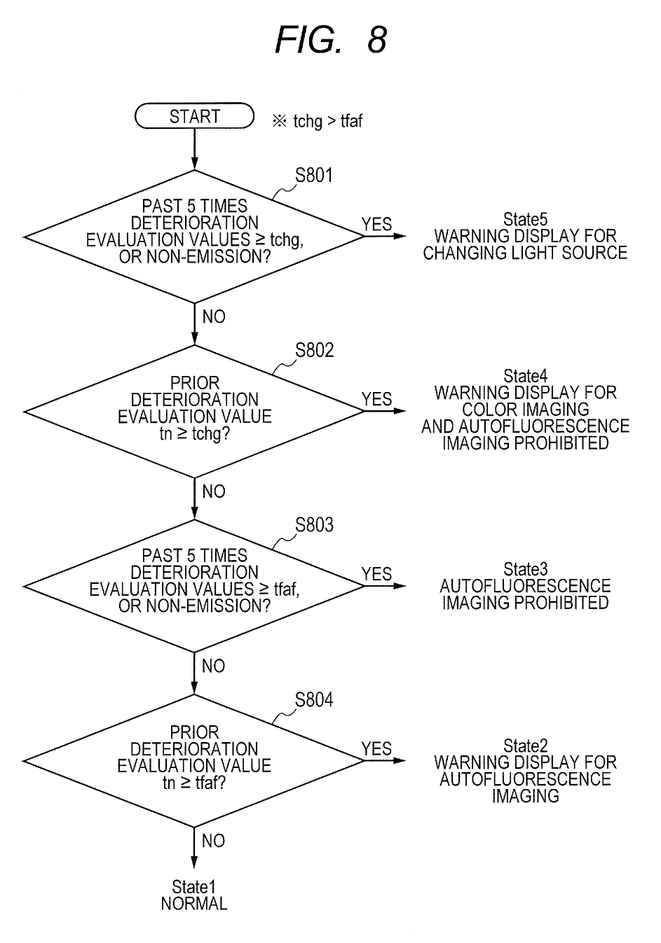

FIG. 8 is a flowchart for determining the deterioration status of the light source on the basis of the deterioration evaluated value and the non-emission detection result according to the second embodiment. The CPU 29 determines the deterioration status following the determination flowchart illustrated in FIG. 8. When all deterioration determination steps are cleared, the deterioration status is determined to be State 1 in which it is determined that there is no deterioration, and the warning display, the imaging prohibition, and the like are not performed.

When it falls into a condition of Step S804 in which the latest deterioration evaluated value exceeds t_{faf}, the deterioration status is determined to be State 2 in which the autofluorescence imaging cannot be performed if the light intensity is deteriorated further.

When it falls into a condition of Step S803 in which the past five deterioration evaluated values are the non-emission or exceed t_{faf}, the deterioration status is determined to be State 3 in which the autofluorescence imaging cannot be performed in a stable manner.

When it falls into a condition of Step S802 in which the latest deterioration evaluated value exceeds t_{chg}, the deterioration status is determined to be State 4 in which not only the autofluorescence imaging but also the color imaging cannot be performed if the light intensity is deteriorated further.

When it falls into a condition of Step S801 in which the past five deterioration evaluated values are the non-emission or exceed t_{chg}, the deterioration status is determined to be State 5 in which the light source is considerably deteriorated so that the imaging cannot be performed in a stable manner.

That is, in the second embodiment, the light source deterioration detection unit records the time from the start of the emission of the imaging light source until the light intensity reaches the reference light intensity for each imaging to use the time as the evaluation value, and performs the determination of the degree of deterioration over years on the basis of the evaluation value. In this case, the comparator 46 constitutes a non-emission determination unit configured to determine a non-emission of the imaging light source from a fact that the imaging light intensity fails to reach the reference light intensity within the reference time. Further, the failure of the imaging due to the non-emission of the imaging light source is notified to the user by the display unit 20 that constitutes a failure notifying unit in the present invention. In addition, in the second embodiment, the non-emission status is added to the deterioration status of the imaging light source in addition to the degree of deterioration, and the deterioration status is determined by a module area that functions as a deterioration status determination unit in the CPU 29.

In the configuration described in the above-mentioned first and second embodiments, the light intensity of the imaging light is directly measured from the xenon tube. However, the imaging light intensity obtained from the xenon tube may be determined on the basis of return light obtained by the imaging light from the xenon tube being irradiated to the eye to be inspected and reflected therefrom. In this case, this mode can be achieved by arranging a light receiving unit configured to receive the return light and a light intensity determination unit configured to detect the light intensity of the return light from the reception result by the light receiving unit and to determine the light intensity of the light from the imaging light source, in addition to the above-mentioned configuration. The above-mentioned first and second embodiments exemplify the embodiment implementing the present invention, and therefore, the embodiments may be achieved by using a known substitute unit having an equivalent function.

### Other Embodiments

Further, the present invention is also implemented by executing the following processing.

Specifically, in this processing, software (program) for implementing the functions of the above-mentioned embodiments is supplied to a system or an apparatus via a network or various kinds of storage medium, and a computer (or CPU, MPU, etc.) of the system or the apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-051451, filed Mar. 8, 2012, which is hereby incorporated by reference herein in its entirety.

What is claimed is:

1. An ophthalmologic apparatus, comprising:
an imaging light source;
a light intensity detection unit configured to detect a light intensity of light emitted from the imaging light source;
a determination unit configured to determine a status of the imaging light source on the basis of the light intensity detected by the light intensity detection unit and a light emitting time of the imaging light source; and
a display control unit configured to display, on a display unit, the status of the imaging light source on the basis of a determination result by the determination unit.

2. An ophthalmologic apparatus according to claim 1, wherein the display control unit is configured to select a display form for indicating the status of the imaging light source and to display the selected display form on the display unit.

3. An ophthalmologic apparatus according to claim 1, wherein the light emitting time of the imaging light source is a time from a start of an emission until the light intensity detected by the light intensity detection unit reaches a predetermined light intensity.

4. An ophthalmologic apparatus according to claim 1, wherein when the determination unit determines that the light emitting time of the imaging light source has exceeded a predetermined time, the display control unit is configured to display a display form indicating deterioration of the imaging light source on the display unit.

5. An ophthalmologic apparatus according to claim 1, further comprising an imaging prohibiting unit configured to prohibit an autofluorescence imaging when the determination unit determines that the light emitting time of the imaging light source has exceeded a predetermined time and when an autofluorescence imaging mode is selected from among multiple imaging modes for imaging an eye to be inspected,
wherein the display control unit is configured to display a display form indicating the prohibition of the autofluorescence imaging on the display unit.

6. An ophthalmologic apparatus according to claim 1, wherein the determination unit includes:
a recording control unit configured to record the light emitting time of the imaging light source for each emission of the imaging light source in a recording unit; and
a detection unit configured to detect a degree of deterioration of the imaging light source on the basis of the light emitting time of the imaging light source.

7. An ophthalmologic apparatus according to claim 1, further comprising an imaging light source control unit configured to stop the emission of the imaging light source when the light intensity detected by the light intensity detection unit has reached a predetermined light intensity.

8. An ophthalmologic apparatus according to claim 1, further comprising:
a monitor light source;
a light receiving unit configured to receive return light from an eye to be inspected irradiated with the light of the imaging light source; and
a light intensity determination unit configured to determine a predetermined light intensity on the basis of a reception result by the light receiving unit.

9. An ophthalmologic apparatus according to claim 6, wherein the detection unit is configured to record, as an evaluation value for each imaging, the light intensity measured by the light intensity detection unit from a start of the emission of the imaging light source to a reference time, and to determine the degree of deterioration on the basis of the evaluation value.

10. An ophthalmologic apparatus according to claim 6, wherein the detection unit is configured to record, as an evaluation value for each imaging, a time from a start of the emission of the imaging light source until the light intensity reaches a reference light intensity and to determine the degree of deterioration on the basis of the evaluation value.

11. An ophthalmologic apparatus according to claim 1, further comprising a non-emission detection unit configured to detect a non-emission of the imaging light source from a relationship between the light emitting time of the imaging light source and a light intensity detection result by the light intensity detection unit.

12. An ophthalmologic apparatus according to claim 11, further comprising a non-emission determination unit configured to determine the non-emission of the imaging light source when an imaging light intensity fails to reach a reference light intensity from a start of the emission of the imaging light source to a reference time.

13. An ophthalmologic apparatus according to claim 11, further comprising a failure notifying unit configured to notify a user of a failure of an imaging when the non-emission is detected.

14. An ophthalmologic apparatus according to claim 9, further comprising a deterioration status determination unit configured to determine a deterioration status from a record of the non-emission and the degree of deterioration detected by the detection unit.

15. A method of controlling an ophthalmologic apparatus, comprising:
detecting a light intensity of light emitted from an imaging light source;
determining a status of the imaging light source on the basis of the light intensity detected in the detecting and a light emitting time of the imaging light source; and
displaying, on a display unit, the status of the imaging light source on the basis of a determination result in the determining.

16. A method of controlling an ophthalmologic apparatus according to claim 15, wherein the displaying comprises selecting a display form for indicating the status of the imaging light source and displaying the selected display form on the display unit.

17. A non-transiently medium storing a program for causing a computer to execute the steps of the method of controlling an ophthalmologic apparatus according to claim 15.
